(19) United States
(12) Patent Application Publication
(10) Pub. No.: US 2001/0051794 A1
(43) Pub. Date: Dec. 13, 2001

(54) Port body for the administration of drugs

(76) Inventors: Gilberto Bestetti, Koinz (CH); Thomas Frei, Lutzelfuh (CH); Andreas Reinmann, Bern (CH); Daniel Piller, Bern (CH)

Correspondence Address: David E. Bruhn, Dorsey & Whitney LLP, 220 South Sixth Street, Minneapolis, MN, 55402-1498, US

(21) Appl. No.: 09/923,122

(22) Filed: Aug. 6, 2001

Related U.S. Application Data
(63) Continuation of application No. 09/048,682, filed on Mar. 26, 1998, now Pat. No. 6,270,475.

(30) Foreign Application Priority Data
Mar. 26, 1997 (CH) 1997 0729/97

(51) Int. Cl. A61M 37/00; A61M 31/00

(52) U.S. Cl. 604/288.04; 604/95.05

(57) ABSTRACT

Implantable cylindrical device for connecting a hose arranged outside of the human or animal body to a hose arranged inside of the said body, wherein an anchoring plate falling away peripherally from the skin surface is arranged around the cylindrical device. This allows the skin to unroll over the anchoring plate subject to a tilting moment of the port body.

### PRIORITY CLAIM

This application claims priority of Swiss patent application 1997 0729197, filed Mar. 26, 1997, which is hereby fully incorporated herein by reference.

### BACKGROUND OF THE INVENTION

1. Field of the Invention

The invention refers to an implantable cylindrical device for connecting a hose outside of the human or animal body to a hose arranged inside the said body, wherein an anchoring plate falling away peripherally from the skin surface is arranged around the cylindrical device.

2. Description of the Related Art

U.S. Pat. No. 5,306,255 describes a subcutaneous implantable port body. A subcutaneous port body is covered completely by skin and normally remains inside the human body for several months or even years. The port body contains the port chamber. The port chamber, fully located inside the human or animal body, is sealed by a puncturable plastic membrane on the skin side, while a catheter leading to the drug release site is fixed on the side facing the interior of the body. In order to administer the drugs, the skin and membrane are punctured with a needle of an infusion set. The creates a continuous drug channel from the infusion set to the release site.

European Patent EP-B-0 302 076 describes a cylindrical, percutaneous implantable port body. In contrast to the subcutaneous port body, the percutaneous port body is not fully implanted in the human body but is fixed in the tissue in such a way that at least a certain area of the port body is not covered by skin. The center of this area contains a first aperture. A second aperture of the port body is located opposite the first aperture in the section of the port body completely surrounded by tissue. A catheter whose end is located at the site inside the body to which the drug is to be transported is connected to this aperture. The port body consists of two metal parts which are screwed together. The inside of the port body, the port chamber, contains a puncturable membrane, separating the two apertures. The external casing of the port body contains several radial grooves for laterally achoring the port in the subcutaneous skin tissue, with the outermost groove being located directly underthe surface of the skin. The port chamber is also anchored with a base plate in the tissue.

The disadvantages of the subcutaneous port are that the catheter can neither be changed nor mechanically cleaned without explanting the port. A further disadvantage is that the skin is always punctured in the same place. In the short term this is painful and in the long term this causes a perforation of the skin and membrane.

The disadvantages of the described percutaneous port are that it is very heavy and has a large visible external surface. The metal port body is furthermore easily noticeable because of its color. Installed port bodies contain a gap between the base plate and the port body which is difficult to clean and sterilize. This represents an infection hazard. The radial grooves are arranged and dimensioned in such a way that sharp edges and corners are created. In these areas an effective growing-in of the tissue cells and adequate cleaning of the surface is not possible. Due to a lack of a geometrical separating line between the skin surface and the uppermost groove, external body perspiration or dirt may directly enter the grooves. In extreme cases this may cause an infection and require the port to be explanted. A further disadvantage is that the components of the described port must be machined from solid material. The manufacturing costs are consequently high with any weight reduction measures incurring additional costs. Prior art anchorings also present the hazard that parts of the anchoring may project from the skin due to the effect of a tilting moment.

### SUMMARY OF THE INVENTION

The invention aims to remedy this situation. It is the aim of the invention to develop a low-cost port whose housing is adapted to the body-shape and contains an interconnected casing surface and continuous transitional areas. The port should preferably be produced by injection molding and be biocompatible. The skin should be able to grow tightly around the port wall. The growing-in depths of the skin should be as even as possible and should be controllable from the port. The shape of the anchoring must be designed in such a way that no edges or other parts of the anchoring protrude from the patient's body in case of a tilting moment.

The invention solves the set task by providing an implantable cylindrical device for connecting a hose outside of the human or animal body to a hose arranged inside the said body, wherein an anchoring plate falling away peripherally from the skin surface is arranged around the cylindrical device.

The invention offers the principle advantages of producing a cheaper port body which can be cleaned better when in use, is retained better by the body due to the design of its external surface and rolls the skin over the anchoring areas in case of a tilting moment. The selected material, shape and surface structure of the port body facilitate a longer implantation period.

### BRIEF DESCRIPTION OF THE DRAWINGS

A preferred embodiment of the invention is shown in the figures, in which:

FIG. 1 represents a cross section of the port body according to the invention; and

FIG. 2 represents a percutaneous port body according to the invention, located in a human or animal body.

### DESCRIPTION OF THE PREFERRED EMBODIMENTS

Hereinafter the term “inside” will mean “within the human or animal body” and “outside” will mean “outside of the human or animal body.”

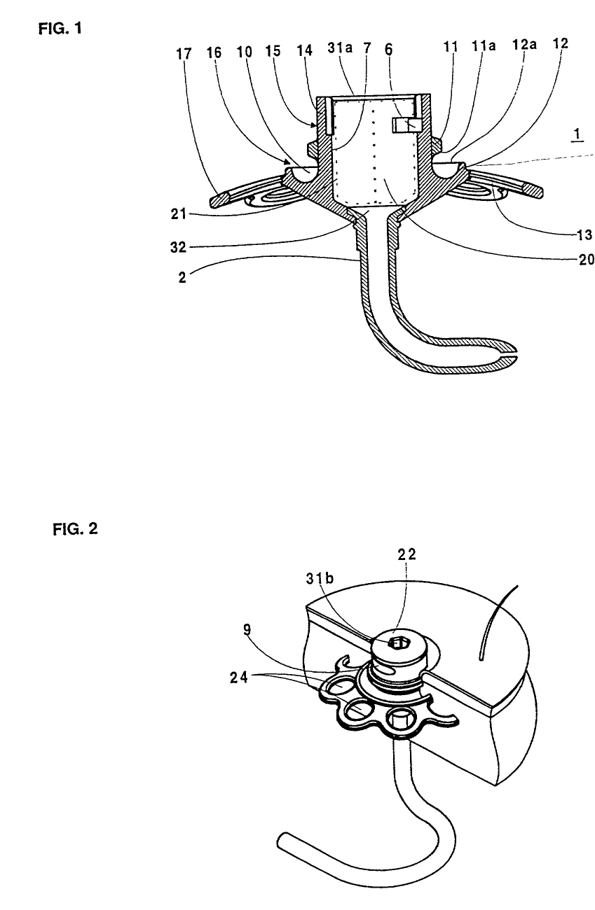

As shown in FIGS. 1 and 2, the port body 1 can be divided into two main elements: a hollow cylindrical shaft 14 and a radial anchoring plate 13 arranged on the said shaft.

The port body 1 contains two opposing apertures 31a, 32. Aperture 31a, facing towards the outside, corresponds to the internal diameter of the cylindrical port body 1. This opening 31a can be decreased in size by a lid 22 containing a smaller aperture 31b in its center. An infusion hose can be pushed through this small remaining aperture 31b into the inside of the port body. The second aperture 32, facing towards the inside, serves to arrange a catheter 2 which moves the drug to be administered to the desired site inside the body.

In the area of the lid 22 the internal wall 7 of the cylindrical port body 1 contains bayonet cams 6 with an integrated locking groove, allowing the lid 22, containing corresponding counter-elements, to be secured to the port body.

The hollow cylindrical shaft 14 and the anchoring plate 13 are molded from a single biologically compatible plastic component. A flexible, self-closing membrane 21 is arranged between the two apertures 31b and 32 filling and sealing the chamber 20 formed by the hollow cylindrical shaft 14.

The cylindrical port body 14 is divided into two areas, a shaft part 15, facing towards the outside, and an anchoring part 16, facing towards the inside. A protruding port fin 11 is radially arranged in between the two areas 15, 16. 6 The shaft part 15 is made of an inert material with a smooth surface structure. It ends in the outward facing aperture 31 on the side facing towards the outside and with the protruding port fin 11 on the side facing the anchoring part 16. In this area the skin cannot grow. When implanted, the shaft part 15 can be cleaned up to the port fin 11 from outside.

The anchoring part 16 consists of the port fin 11, an anchoring fin 12 protruding from the anchoring part 16 and the anchoring plate 13. Both the port fin 11 and the anchoring fin 12 contain a peripheral fin edge 11a, 12a. A channel-shaped, radial pocket 10 is formed between the port fin 11 and the anchoring fin 12 as the gap between the two peripheral fin edges 11a, 12a is considerably smaller than the cross-sectional diameter of the radial pocket 10 itself. Due to the gap formed between the two fin edges 11a, 12a, tissue cells can grow into the channel-shaped radial pocket 10.

The anchoring rib 12 may be part of the anchoring plate 13 or may be arranged separately from the said plate between the port fin 11 and the anchoring plate 13.

The anchoring part 16 is coated with a bio-active material and has a rough structure. This allows tissue to spread inside the pocket 10, and tissue cells can attached themselves to the rough surface.

During the growth of the tissue into the radial pocket 10, the tissue wedges itself in the radial pocket 10 and ensures a flush connection between the tissue and the surface of the radial pocket 10.

The anchoring plate 13 is radially arranged around the anchoring part 16 of the hollow cylindrical shaft 14. The anchoring plate 13 has a plate-like shape falling away peripherally from the skin surface. During the effect of a tilting moment on the port body 1, the falling-away shape causes the skin to roll over the anchoring plate 13 instead of being pierced by its edge 17.

The anchoring plate 13 contains holes 24 through which the surround tissue grows to offer maximum retention.

In order to be able to open the port lid 22 once implanted, an installation aid recess 9 is arranged at the top end of the outer surface of the port shaft 15. A special tool grips into three such recesses 9 arranged at the same level, and the lid 22, whose aperture 31b is of a hexagonal shape, is released from its connection with the port body 14 by turning.

In a percutaneous port body 1 the lid 22 is preferably produced in a skin-like color as this area is visible from the outside. Naturally the entire port body may be produced in a skin-like color.

We claim:

1. Implantable cylindrical device for connecting a hose outside of the human or animal body to a hose arranged inside the said body, characterized in that an anchoring plate falling away peripherally from the skin surface is arranged around the cylindrical device.

2. Device according to claim 1, characterized in that one or several radial fins protrude from the external casing of the cylindrical device.

3. Device according to claim 1, characterized in that one or several fins protrude from the anchoring plate.

4. Device according to claim 3, characterized in that the distance between the two fin edges is smaller than the cross-sectional diameter of the channel-shaped radial pocket formed by the fins.

5. Device according to claim 4, characterized in that the device can be a molded plastic component.

6. Device according to claim 5, characterized in that the device or parts thereof have a color similar to that of the skin.

7. Device according to claim 6, characterized in that the device is covered completely by the skin.

8. Device according to claim 6, characterized in that a surface containing an aperture is not arranged within the body.

9. Device according to claim 7, characterized in that the hose arranged inside the human or animal body seals an aperture of the device and a lid containing a small aperture in its center, reduces in size a second aperture of the device.

10. A device according to claim 9, characterized in that a puncturable, flexible membrane sealing the second aperture is arranged inside the device.

11. Device according to claim 2, characterized in that the distance between the two fin edges is smaller than the cross-sectional diameter of the channel-shaped radial pocket formed by the fins.

12. Device according to claim 11, characterized in that the device can be a molded plastic component.

13. Device according to claim 12, characterized in that the device or parts thereof have a color similar to that of the skin.

14. Device according to claim 13, characterized in that the device is covered completely by the skin.

15. Device according to claim 13, characterized in that a surface containing an aperture is not arranged within the body.

16. Device according to claim 15, characterized in that the hose arranged inside the human or animal body seals an aperture of the device and a lid containing a small aperture in its center, reduces in size a second aperture of the device.

17. A device according to claim 16, characterized in that a puncturable, flexible membrane sealing the second aperture is arranged inside the device.

18. Device according to claim 14, characterized in that the hose arranged inside the human or animal body seals an aperture of the device and a lid containing a small aperture in its center, reduces in size a second aperture of the device.

19. A device according to claim 18, characterized in that a puncturable, flexible membrane sealing the second aperture is arranged inside the device.
